(19) United States
(12) Patent Application Publication
(10) Pub. No.: US 2013/0197017 A1
(43) Pub. Date: Aug. 1, 2013

(54) COMBINATIONS COMPRISING A PROTEIN KINASE INHIBITOR BEING A PYRIMIDYLAMINOBENZAMIDE COMPOUND AND A HSP90 INHIBITOR SUCH AS 17-11G

(71) Applicants: Paul W Manley, Arlesheim (CH); Kapil N Bhalla, Tampa, FL (US)

(72) Inventors: Paul W Manley, Arlesheim (CH); Kapil N Bhalla, Tampa, FL (US)

(21) Appl. No.: 13/793,490

(22) Filed: Mar. 11, 2013

Related U.S. Application Data
(63) Continuation of application No. 13/195,369, filed on Aug. 1, 2011.
Application No. 13/195,369 is a continuation of application No. 12/063,164, filed on Feb. 7, 2008, now abandoned.
(60) Provisional application No. 60/707,435, filed on Aug. 11, 2005.
(60) Provisional application No. 60/730,689, filed on Oct. 27, 2005.

(51) Int. Cl. A61K 31/506; A61K 31/365; A61K 45/06; A61K 31/395

(52) U.S. Cl. CPC A61K 31/506; A61K 31/395; A61K 31/365; A61K 45/06
USPC 514/275

(57) ABSTRACT

The invention provides a pharmaceutical combination comprising:
a) a pyrimidylaminobenzamide compound; and
b) an HSP90 inhibitor,
   and a method for treating or preventing a proliferative disease using such a combination.

The present invention relates to a pharmaceutical combination comprising a pyrimidylaminobenzamide compound and inhibitors of Heat Shock Proteins, such as HSP70 and HSP90, and the uses of such combinations, e.g., in proliferative diseases, tumors, myelomas, leukemias, psoriasis, restenosis, sclerodermitis and fibrosis.

In spite of numerous treatment options for proliferative disease patients, there remains a need for effective and safe antiproliferative agents and a need for their preferential use in combination therapy.

The HSP90 family of chaperones is comprised of four known members: HSP90α and HSP90β both in the cytosol, grp94 in the endoplasmic reticulum and trap-1 in the mitochondria. HSP90 is an abundant cellular chaperone required for the ATP-dependent refolding of denatured or “unfolded” proteins and for the conformational maturation of a variety of key proteins involved in the growth response of the cell to extracellular factors. These proteins, which are called client proteins, include the steroid receptors, as well as various protein kinases. HSP90 is essential for eukaryotic cell survival and is over-expressed in many tumors. Cancer cells show sensitivity to transient inhibition of HSP90 ATPase activity indicating that HSP90 inhibitors could have a potential as new anticancer drugs. Each HSP90 family member possesses a conserved ATP-binding site at its N-terminal domain, which is found in few other ATP-binding proteins. The weak ATPase activity of HSP90 is stimulated upon its interaction with various co-chaperone proteins. Several natural compounds, such as geldanamycin or radicicol bind at the ATP-binding site of HSP90 inhibiting its ATPase activity. In cellular systems and in vivo, these drugs upon binding to HSP90 prevent the folding of the client proteins, which are then degraded in the proteasome. 17-allylamino-17-demethoxygeldanamycin (17-AAG), a geldanamycin derivative, is currently undergoing Phase I and Phase II clinical trials in hematological malignancies and solid tumors at several institutions. Initial clinical experiences with 17-MG have offered preliminary evidence that concentrations of the drug associated with activity in pre-clinical systems can be achieved in humans with tolerable toxicity, and provided early evidence of target modulation in at least certain surrogate and tumor compartments. The dose limiting toxicity of 17-MG is hepatic. 17-MG poor solubility makes it difficult to formulate/administer and its synthesis is difficult (it is generally obtained by fermentation).

### SUMMARY OF THE INVENTION

It has now been found that a combination comprising at least one pyrimidylaminobenzamide compound and an HSP90 inhibitor, e.g., as defined below, has a beneficial effect on proliferative diseases, e.g., tumors, myelomas, leukemias, psoriasis, restenosis, sclerodermitis and fibrosis.

### DETAILED DESCRIPTION OF THE INVENTION

The present invention relates to the use of pyrimidylaminobenzamide compounds of formula (I):

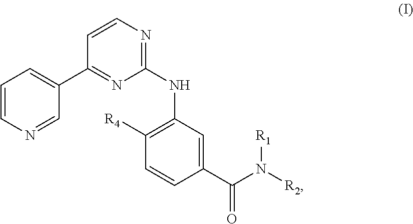

wherein
R₁ represents hydrogen, lower alkyl, lower alkoxy-lower alkyl, acyloxy-lower alkyl, carboxy-lower alkyl, lower alkoxycarbonyl-lower alkyl or phenyl-lower alkyl;
R₂ represents hydrogen, lower alkyl, optionally substituted by one or more identical or different radicals R₃, cycloalkyl, benzcycloalkyl, heterocyclyl, an aryl group, or a mono- or bicyclic heteroaryl group comprising zero, one, two or three ring nitrogen atoms and zero or one oxygen atom and zero or one sulfur atom, which groups in each case are unsubstituted or mono- or polysubstituted;
R₃ represents hydroxy, lower alkoxy, acyloxy, carboxy, lower alkoxycarbonyl, carbamoyl, N-mono- or N,N-disubstituted carbamoyl, amino, mono- or disubstituted amino, cycloalkyl, heterocyclyl, an aryl group, or a mono- or bicyclic heteroaryl group comprising zero, one, two or three ring nitrogen atoms and zero or one oxygen atom and zero or one sulfur atom, which groups in each case are unsubstituted or mono- or polysubstituted, or
R₁ and R₂ together represent alkylene with four, five or six carbon atoms optionally mono- or disubstituted by lower alkyl, cycloalkyl, heterocyclyl, phenyl, hydroxy, lower alkoxy, amino, mono- or disubstituted amino, oxo, pyridyl, pyrazinyl or pyrimidinyl; benzalkylene with four or five carbon atoms; oxaalkylene with one oxygen and three or four carbon atoms; or azaalkylene with one nitrogen and three or four carbon atoms wherein nitrogen is unsubstituted or substituted by lower alkyl, phenyl-lower alkyl, lower alkoxycarbonyl-lower alkyl, carboxy-lower alkyl, carbamoyl-lower alkyl, N-mono- or N,N-disubstituted carbamoyl-lower alkyl, cycloalkyl, lower alkoxycarbonyl, carboxy, phenyl, substituted phenyl, pyridinyl, pyrimidinyl, or pyrazinyl;
R₄ represents hydrogen, lower alkyl, or halogen; and
   a N-oxide or a pharmaceutically acceptable salt of such a compound for the preparation of a pharmaceutical composition for the treatment of kinase dependent diseases.

The general terms used hereinbefore and hereinafter preferably have within the context of this disclosure the following meanings, unless otherwise indicated:

The prefix “lower” denotes a radical having up to and including a maximum of 7, especially up to and including a maximum of 4 carbon atoms, the radicals in question being either linear or branched with single or multiple branching.

Where the plural form is used for compounds, salts, and the like, this is taken to mean also a single compound, salt, or the like.

Any asymmetric carbon atoms may be present in the (R)-, (S)- or (R,S)-configuration, preferably in the (R)- or (S)-configuration. The compounds may thus be present as mixtures of isomers or as pure isomers, preferably as enantiomer-pure diastereomers.

The invention relates also to possible tautomers of the compounds of formula (I).

Lower alkyl is preferably alkyl with from and including 1 up to and including 7, preferably from and including 1 to and including 4, and is linear or branched; preferably, lower alkyl is butyl, such as n-butyl, sec-butyl, isobutyl, tert-butyl, propyl, such as n-propyl or isopropyl, ethyl or methyl. Preferably lower alkyl is methyl, propyl or tert-butyl.

Lower acyl is preferably formyl or lower alkylcarbonyl, in particular, acetyl.

An aryl group is an aromatic radical which is bound to the molecule via a bond located at an aromatic ring carbon atom of the radical. In a preferred embodiment, aryl is an aromatic radical having 6 to 14 carbon atoms, especially phenyl, naphthyl, tetrahydronaphthyl, fluorenyl or phenanthrenyl, and is unsubstituted or substituted by one or more, preferably up to three, especially one or two substituents, especially selected from amino, mono- or disubstituted amino, halogen, lower alkyl, substituted lower alkyl, lower alkenyl, lower alkynyl, phenyl, hydroxy, etherified or esterified hydroxy, nitro, cyano, carboxy, esterified carboxy, alkanoyl, benzoyl, carbamoyl, N-mono- or N,N-disubstituted carbamoyl, amidino, guanidino, ureido, mercapto, sulfo, lower alkylthio, phenylthio, phenyl-lower alkylthio, lower alkylphenylthio, lower alkylsulfinyl, phenylsulfinyl, phenyl-lower alkylsulfinyl, lower alkylphenylsulfinyl, lower alkylsulfonyl, phenylsulfonyl, phenyl-lower alkylsulfonyl, lower alkylphenylsulfonyl, halogen-lower alkylmercapto, halogen-lower alkylsulfonyl, such as especially trifluoromethanesulfonyl, dihydroxybora (—B(OH)₂), heterocyclyl, a mono- or bicyclic heteroaryl group and lower alkylene dioxy bound at adjacent C-atoms of the ring, such as methylene dioxy. Aryl is more preferably phenyl, naphthyl or tetrahydronaphthyl, which in each case is either unsubstituted or independently substituted by one or two substituents selected from the group comprising halogen, especially fluorine, chlorine, or bromine; hydroxy; hydroxy etherified by lower alkyl, e.g., by methyl, by halogen-lower alkyl, e.g., trifluoromethyl, or by phenyl; lower alkylene dioxy bound to two adjacent C-atoms, e.g., methylenedioxy, lower alkyl, e.g., methyl or propyl; halogen-lower alkyl, e.g., trifluoromethyl; hydroxy-lower alkyl, e.g., hydroxymethyl or 2-hydroxy-2-propyl; lower alkoxy-lower alkyl; e.g., methoxymethyl or 2-methoxyethyl; lower alkoxycarbonyl-lower alkyl, e.g., methoxycarbonylmethyl; lower alkynyl, such as 1-propynyl; esterified carboxy, especially lower alkoxycarbonyl, e.g., methoxycarbonyl, n-propoxy carbonyl or iso-propoxy carbonyl; N-mono-substituted carbamoyl, in particular carbamoyl monosubstituted by lower alkyl, e.g., methyl, n-propyl or iso-propyl; amino; lower alkylamino, e.g., methylamino; di-lower alkylamino, e.g., dimethylamino or diethylamino; lower alkylene-amino, e.g., pyrrolidino or piperidino; lower oxaalkylene-amino, e.g., morpholino, lower azaalkylene-amino, e.g., piperazino, acylamino, e.g., acetylamino or benzoylamino; lower alkylsulfonyl, e.g., methylsulfonyl; sulfamoyl; or phenylsulfonyl.

A cycloalkyl group is preferably cyclopropyl, cyclopentyl, cyclohexyl or cycloheptyl, and may be unsubstituted or substituted by one or more, especially one or two, substituents selected from the group defined above as substituents for aryl, most preferably by lower alkyl, such as methyl, lower alkoxy, such as methoxy or ethoxy, or hydroxy, and further by oxo or fused to a benzo ring, such as in benzcyclopentyl or benzcyclohexyl.

Substituted alkyl is alkyl as last defined, especially lower alkyl, preferably methyl; where one or more, especially up to three, substituents may be present, primarily from the group selected from halogen, especially fluorine, amino, N-lower alkylamino, N,N-di-lower alkylamino, N-lower alkanoylamino, hydroxy, cyano, carboxy, lower alkoxycarbonyl, and phenyl-lower alkoxycarbonyl. Trifluoromethyl is especially preferred.

Mono- or disubstituted amino is especially amino substituted by one or two radicals selected independently of one another from lower alkyl, such as methyl; hydroxy-lower alkyl, such as 2-hydroxyethyl; lower alkoxy lower alkyl, such as methoxy ethyl; phenyl-lower alkyl, such as benzyl or 2-phenylethyl; lower alkanoyl, such as acetyl; benzoyl; substituted benzoyl, wherein the phenyl radical is especially substituted by one or more, preferably one or two, substituents selected from nitro, amino, halogen, N-lower alkylamino, N,N-di-lower alkylamino, hydroxy, cyano, carboxy, lower alkoxycarbonyl, lower alkanoyl, and carbamoyl; and phenyl-lower alkoxycarbonyl, wherein the phenyl radical is unsubstituted or especially substituted by one or more, preferably one or two, substituents selected from nitro, amino, halogen, N-lower alkylamino, N,N-di-lower alkylamino, hydroxy, cyano, carboxy, lower alkoxycarbonyl, lower alkanoyl, and carbamoyl; and is preferably N-lower alkylamino, such as N-methylamino, hydroxy-lower alkylamino, such as 2-hydroxyethylamino or 2-hydroxypropyl, lower alkoxy lower alkyl, such as methoxy ethyl, phenyl-lower alkylamino, such as benzylamino, N,N-di-lower alkylamino, N-phenyl-lower alkyl-N-lower alkylamino, N,N-di-lower alkylphenylamino, lower alkanoylamino, such as acetylamino, or a substituent selected from the group comprising benzoylamino and phenyl-lower alkoxycarbonylamino, wherein the phenyl radical in each case is unsubstituted or especially substituted by nitro or amino, or also by halogen, amino, N-lower alkylamino, N,N-di-lower alkylamino, hydroxy, cyano, carboxy, lower alkoxycarbonyl, lower alkanoyl, carbamoyl or aminocarbonylamino. Disubstituted amino is also lower alkylene-amino, e.g., pyrrolidino, 2-oxopyrrolidino or piperidino; lower oxaalkylene-amino, e.g. morpholino, or lower azaalkylene-amino, e.g., piperazino or N-substituted piperazino, such as N-methylpiperazino or N-methoxycarbonylpiperazino.

Halogen is especially fluorine, chlorine, bromine, or iodine, especially fluorine, chlorine, or bromine.

Etherified hydroxy is especially C₈-C₂₀alkyloxy, such as n-decyloxy, lower alkoxy (preferred), such as methoxy, ethoxy, isopropyloxy, or tert-butyloxy, phenyl-lower alkoxy, such as benzyloxy, phenyloxy, halogen-lower alkoxy, such as trifluoromethoxy, 2,2,2-trifluoroethoxy or 1,1,2,2-tetrafluoroethoxy, or lower alkoxy which is substituted by mono- or bicyclic heteroaryl comprising one or two nitrogen atoms, preferably lower alkoxy which is substituted by imidazolyl, such as 1H-imidazol-1-yl, pyrrolyl, benzimidazolyl, such as 1-benzimidazolyl, pyridyl, especially 2-, 3- or 4-pyridyl, pyrimidinyl, especially 2-pyrimidinyl, pyrazinyl, isoquinolinyl, especially 3-isoquinolinyl, quinolinyl, indolyl or thiazolyl.

Esterified hydroxy is especially lower alkanoyloxy, benzoyloxy, lower alkoxycarbonyloxy, such as tert-butoxycarbonyloxy, or phenyl-lower alkoxycarbonyloxy, such as benzyloxycarbonyloxy.

Esterified carboxy is especially lower alkoxycarbonyl, such as Pert-butoxycarbonyl, iso-propoxycarbonyl, methoxycarbonyl or ethoxycarbonyl, phenyl-lower alkoxycarbonyl, or phenyloxycarbonyl.

Alkanoyl is primarily alkylcarbonyl, especially lower alkanoyl, e.g., acetyl.

N-Mono- or N,N-disubstituted carbamoyl is especially substituted by one or two substituents independently selected from lower alkyl, phenyl-lower alkyl and hydroxy-lower alkyl, or lower alkylene, oxa-lower alkylene or aza-lower alkylene optionally substituted at the terminal nitrogen atom.

A mono- or bicyclic heteroaryl group comprising zero, one, two or three ring nitrogen atoms and zero or one oxygen atom and zero or one sulfur atom, which groups in each case are unsubstituted or mono- or polysubstituted, refers to a heterocyclic moiety that is unsaturated in the ring binding the heteroaryl radical to the rest of the molecule in formula (I) and is preferably a ring, where in the binding ring, but optionally also in any annealed ring, at least one carbon atom is replaced by a heteroatom selected from the group consisting of nitrogen, oxygen and sulfur; where the binding ring preferably has 5 to 12, more preferably 5 or 6 ring atoms; and which may be unsubstituted or substituted by one or more, especially one or two, substituents selected from the group defined above as substituents for aryl, most preferably by lower alkyl, such as methyl, lower alkoxy, such as methoxy or ethoxy, or hydroxy. Preferably the mono- or bicyclic heteroaryl group is selected from 2H-pyrrolyl, pyrrolyl, imidazolyl, benzimidazolyl, pyrazolyl, indazolyl, purinyl, pyridyl, pyrazinyl, pyrimidinyl, pyridazinyl, 4H-quinolizinyl, isoquinolyl, quinolyl, phthalazinyl, naphthyridinyl, quinoxalyl, quinazolinyl, quinnolinyl, pteridinyl, indolizinyl, 3H-indolyl, indolyl, isoindolyl, oxazolyl, isoxazolyl, thiazolyl, isothiazolyl, triazolyl, tetrazolyl, furazanyl, benzo[d]pyrazolyl, thienyl and furanyl. More preferably the mono- or bicyclic heteroaryl group is selected from the group consisting of pyrrolyl, imidazolyl, such as 1H-imidazol-1-yl, benzimidazolyl, such as 1-benzimidazolyl, indazolyl, especially 5-indazolyl, pyridyl, especially 2-, 3- or 4-pyridyl, pyrimidinyl, especially 2-pyrimidinyl, pyrazinyl, isoquinolinyl, especially 3-isoquinolinyl, quinolinyl, especially 4- or 8-quinolinyl, indolyl, especially 3-indolyl, thiazolyl, benzo[d]pyrazolyl, thienyl, and furanyl. In one preferred embodiment of the invention the pyridyl radical is substituted by hydroxy in ortho position to the nitrogen atom and hence exists at least partially in the form of the corresponding tautomer which is pyridin-(1H)₂-one. In another preferred embodiment, the pyrimidinyl radical is substituted by hydroxy both in position 2 and 4 and hence exists in several tautomeric forms, e.g., as pyrimidine-(1H, 3H)2,4-dione.

Heterocyclyl is especially a five, six or seven-membered heterocyclic system with one or two heteroatoms selected from the group comprising nitrogen, oxygen, and sulfur, which may be unsaturated or wholly or partly saturated, and is unsubstituted or substituted especially by lower alkyl, such as methyl, phenyl-lower alkyl, such as benzyl, oxo, or heteroaryl, such as 2-piperazinyl; heterocyclyl is especially 2- or 3-pyrrolidinyl, 2-oxo-5-pyrrolidinyl, piperidinyl, N-benzyl-4-piperidinyl, N-lower alkyl-4-piperidinyl, N-lower alkyl-piperazinyl, morpholinyl, e.g., 2- or 3-morpholinyl, 2-oxo-1H-azepin-3-yl, 2-tetrahydrofuranyl, or 2-methyl-1,3-dioxolan-2-yl.

Salts are especially the pharmaceutically acceptable salts of compounds of formula (I).

Such salts are formed, e.g., as acid addition salts, preferably with organic or inorganic acids, from compounds of formula (I) with a basic nitrogen atom, especially the pharmaceutically acceptable salts. Suitable inorganic acids are, e.g., halogen acids, such as hydrochloric acid, sulfuric acid, or phosphoric acid. Suitable organic acids are, e.g., carboxylic, phosphonic, sulfonic or sulfamic acids, e.g., acetic acid, propionic acid, octanoic acid, decanoic acid, dodecanoic acid, glycolic acid, lactic acid, fumaric acid, succinic acid, adipic acid, pimelic acid, suberic acid, azelaic acid, malic acid, tartaric acid, citric acid, amino acids, such as glutamic acid or aspartic acid, maleic acid, hydroxymaleic acid, methylmaleic acid, cyclohexanecarboxylic acid, adamantanecarboxylic acid, benzoic acid, salicylic acid, 4-aminosalicylic acid, phthalic acid, phenylacetic acid, mandelic acid, cinnamic acid, methane- or ethane-sulfonic acid, 2-hydroxyethanesulfonic acid, ethane-1,2-disulfonic acid, benzenesulfonic acid, 2-naphthalenesulfonic acid, 1,5-naphthalene-disulfonic acid, 2-, 3- or 4-methylbenzenesulfonic acid, methylsulfuric acid, ethylsulfuric acid, dodecylsulfuric acid, N-cyclohexylsulfamic acid, N-methyl-, N-ethyl- or N-propyl-sulfamic acid, or other organic protonic acids, such as ascorbic acid.

In the presence of negatively charged radicals, such as carboxy or sulfo, salts may also be formed with bases, e.g., metal or ammonium salts, such as alkali metal or alkaline earth metal salts, e.g., sodium, potassium, magnesium or calcium salts, or ammonium salts with ammonia or suitable organic amines, such as tertiary monoamines, e.g., triethylamine or tri(2-hydroxyethyl)amine, or heterocyclic bases, e.g., N-ethyl-piperidine or N,N′-dimethylpiperazine.

When a basic group and an acid group are present in the same molecule, a compound of formula (I) may also form internal salts.

For isolation or purification purposes it is also possible to use pharmaceutically unacceptable salts, e.g., picrates or perchlorates. For therapeutic use, only pharmaceutically acceptable salts or free compounds are employed (where applicable in the form of pharmaceutical preparations), and these are therefore preferred.

In view of the close relationship between the novel compounds in free form and those in the form of their salts, including those salts that can be used as intermediates, e.g., in the purification or identification of the novel compounds, any reference to the free compounds hereinbefore and hereinafter is to be understood as referring also to the corresponding salts, as appropriate and expedient.

Compounds within the scope of formula (I) and the process for their manufacture are disclosed in WO 04/005281 published on Jan. 15, 2004 which is hereby incorporated into the present application by reference. A preferred compound is 4-methyl-3-[[4-(3-pyridinyl)-2-pyrimidinyl]amino]-N-[5-(4-methyl-1H-imidazol-1-yl)-3-(trifluoromethyl)phenyl]benzamide which has the structure (II):

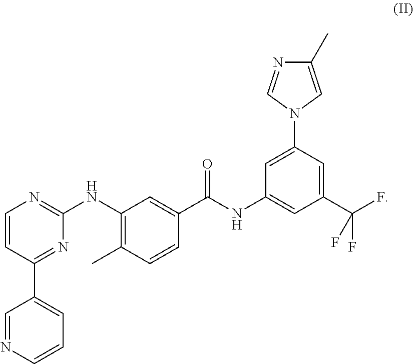 [SMILES: Cc1cn(-c2cc(NC(=O)c3ccc(C)c(Nc4nccc(-c5cccnc5)n4)c3)cc(C(F)(F)F)c2)cn1]

HSP-90 inhibitors are, e.g., compounds having an IC₅₀ value in the range of 1-10,000 nM, preferably in the range of 1-100 nM in the following assay:

The inhibition of HSP90 is measured using the procedure, with minor modifications, described in Schilb et al., J Biomol Screening, Vol. 9, pp. 569-577 (2004).

The compounds of the formula (I) here shown IC₅₀ values in the range between 0.005 and 20 μM, preferably between 0.01 and 10 μM.

Suitable HSP90 inhibitors include, e.g.,

A. The geldanamycin derivative, 17-allylamino-17-demethoxygeldanamycin (17-MG), a geldanamycin derivative, other geldanamycin-related compounds; and Radicicol;
B. Compounds as disclosed in PCT application no. EP2005/008118 filed Jul. 26, 2005, e.g., compound of formula (III):

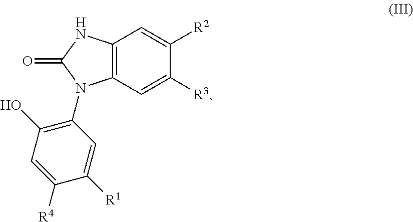

wherein
R¹ is H, halo, substituted or unsubstituted lower alkyl;
R² is H, halo, substituted or unsubstituted lower alkyl, carboxy, COR⁵, SO₂R⁵, CX₂R⁵, CXHR⁵CH₂R⁵, CHR⁵R⁶, CR⁵(R⁶)₂, or C(R⁵)₂R⁶;
R³ is H, substituted or unsubstituted lower alkyl, halo, —SO₂NH₂ or

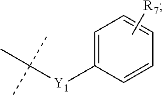

R⁴ is H or hydroxy;
R⁵ is lower alkyl; —(CH₂), —NR⁶₂; —YR⁶; —Y(CH₂), NR⁶₂;

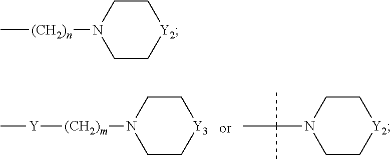

n is 1 or 2;
m is 2 or 3;
X is halo;
Y₁ is alkylene, O, S or N;
Y₂ and Y₃ are each independently methylene, O or NR′;
R⁶ is H, lower alkyl, cycloalkyl, heterocycl, fused cycloalkyl, fused heterocycl or NR⁹R¹⁰ together form a heterocyclic ring with the N atom, form a 3- to 8-membered heterocyclic ring containing 1-4 nitrogen, oxygen or sulfur atoms (e.g. azetidinyl, pyrrolidinyl, piperidino, morpholinyl, imidazolinyl, piperazinyl or lower alkyl-piperazinyl); cycloalkyl as defined above, especially C₃-C₆cycloalkyl, lower alkanoyl (preferably as single amino substituent or in combination with another of the non-acyl moiety just mentioned) and benzoyl or phenyl-lower alkanoyl (preferably as single amino substituent or in combination with another of the non-acyl moiety just mentioned), cyano, cyano-lower alkyl, such as cyanomethyl, amidino, N-hydroxyamidino, amidino-lower alkyl, such as -methyl, or N-hydroxyamidino-lower alkyl, such as -methyl;
R⁷ is lower alkyl, halo, lower alkoxy, or —Y₁—(CH₂)ₚ—N(R⁸)(H);
   p is 1-3;
   R⁸ is H or lower alkyl;
      or pharmaceutically acceptable salts thereof;
      C. Compounds as disclosed in PCT application no. EP2005/008119 filed Jul. 27, 2005, e.g., compound of formula (IV):

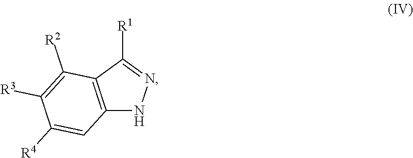

wherein
R¹ is substituted or unsubstituted lower alkyl, substituted or unsubstituted aryl, or substituted or unsubstituted aryl lower alky;
R² is H, halo, hydroxy, lower alkyl or a group of the formula:

—Y—R⁵,
where
   Y is O, N, S or lower alkyl; and
   R⁵ is substituted or unsubstituted lower alkyl, or substituted or unsubstituted aryl;
R³ is H, halo, or substituted or unsubstituted lower alkyl, substituted or unsubstituted aryl, substituted or unsubstituted cycloalkyl, substituted or unsubstituted cycloalkyl-alkyl or substituted or unsubstituted arylalkyl;
R⁴ is H or OH;
   or pharmaceutically acceptable salts thereof.

In each case where citations of patent applications are given above, the subject matter relating to the compounds is hereby incorporated into the present application by reference. Comprised are likewise the pharmaceutically acceptable salts thereof, the corresponding racemates, diastereoisomers, enantiomers, tautomers, as well as the corresponding crystal modifications of above disclosed compounds where present, e.g., solvates, hydrates and polymorphs, which are disclosed therein. The compounds used as active ingredients in the combinations of the invention can be prepared and administered as described in the cited documents, respectively. Also within the scope of this invention is the combination of more than two separate active ingredients as set forth above, i.e., a pharmaceutical combination within the scope of this invention could include three active ingredients or more.

In accordance with the particular findings of the present invention, there is provided:
1. A pharmaceutical combination comprising:
a) a pyrimidylaminobenzamide compound of formula (I); and
b) at least one HSP90 inhibitor.
2. A method for treating or preventing proliferative disease in a subject in need thereof, comprising co-administration to said subject, e.g., concomitantly or in sequence, of a therapeutically effective amount of a pyrimidylaminobenzamide compound of formula (I) and an HSP90 inhibitor, e.g., as disclosed above.
   Examples of proliferative diseases include e.g. tumors, leukemias, psoriasis, restenosis, sclerodermitis and fibrosis.
3. A pharmaceutical combination as defined under 1) above, e.g. for use in a method as defined under 2) above.
4. A pharmaceutical combination as defined under 1) above for use in the preparation of a medicament for use in a method as defined under 2) above.

Utility of the combination of the invention in a method as hereinabove specified, may be demonstrated in animal test methods as well as in clinic, for example in accordance with the methods hereinafter described.

It has now surprisingly been found that the combination of a pyrimidylaminobenzamide compound and a HSP90 inhibitor possesses therapeutic properties, which render it particularly useful as a treatment for proliferative diseases.

In another embodiment, the instant invention provides a method for treating proliferative diseases comprising administering to a mammal in need of such treatment a therapeutically effective amount of the combination of a pyrimidylaminobenzamide compound and an HSP90 inhibitor or pharmaceutically acceptable salts or prodrugs thereof.

Preferably, the instant invention provides a method for treating mammals, especially humans, suffering from proliferative diseases comprising administering to a mammal in need of such treatment an inhibiting amount of the combination of a pyrimidylaminobenzamide compound and an HSP90 inhibitor or pharmaceutically acceptable salts thereof.

In the present description, the term “treatment” includes both prophylactic or preventative treatment, as well as curative or disease suppressive treatment, including treatment of patients at risk of contracting the disease or suspected to have contracted the disease, as well as ill patients. This term further includes the treatment for the delay of progression of the disease.

The term “curative”, as used herein, means efficacy in treating ongoing episodes involving proliferative diseases.

The term “prophylactic” means the prevention of the onset or recurrence of diseases involving proliferative diseases.

The term “delay of progression”, as used herein, means administration of the active compound to patients being in a pre-stage or in an early phase of the disease to be treated, in which patients, e.g., a pre-form of the corresponding disease is diagnosed or which patients are in a condition, e.g., during a medical treatment or a condition resulting from an accident, under which it is likely that a corresponding disease will develop.

This unforeseeable range of properties means that the use of the combination of a pyrimidylaminobenzamide compound and an HSP90 inhibitor are of particular interest for the manufacture of a medicament for the treatment of proliferative diseases.

To demonstrate that the combination of a pyrimidylaminobenzamide compound and an HSP90 inhibitor is particularly suitable for the treatment of proliferative diseases with good therapeutic margin and other advantages, clinical trials can be carried out in a manner known to the skilled person.

### A. Combined Treatment

Suitable clinical studies are, e.g., open label, dose escalation studies in patients with proliferative diseases. Such studies prove in particular the synergism of the active ingredients of the combination of the invention. The beneficial effects can be determined directly through the results of these studies which are known as such to a person skilled in the art. Such studies are, in particular, suitable to compare the effects of a monotherapy using the active ingredients and a combination of the invention. Preferably, the dose of agent (a) is escalated until the Maximum Tolerated Dosage is reached, and agent (b) is administered with a fixed dose. Alternatively, the agent (a) is administered in a fixed dose and the dose of agent (b) is escalated. Each patient receives doses of the agent (a) either daily or intermittent. The efficacy of the treatment can be determined in such studies, e.g., after 12, 18 or 24 weeks by evaluation of symptom scores every 6 weeks.

The administration of a pharmaceutical combination of the invention results not only in a beneficial effect, e.g., a synergistic therapeutic effect, e.g., with regard to alleviating, delaying progression of or inhibiting the symptoms, but also in further surprising beneficial effects, e.g., fewer side effects, an improved quality of life or a decreased morbidity, compared with a monotherapy applying only one of the pharmaceutically active ingredients used in the combination of the invention.

A further benefit is that lower doses of the active ingredients of the combination of the invention can be used, e.g., that the dosages need not only often be smaller but are also applied less frequently, which may diminish the incidence or severity of side effects. This is in accordance with the desires and requirements of the patients to be treated.

The terms “co-administration” or “combined administration” or the like as utilized herein are meant to encompass administration of the selected therapeutic agents to a single patient, and are intended to include treatment regimens in which the agents are not necessarily administered by the same route of administration or at the same time.

It is one objective of this invention to provide a pharmaceutical composition comprising a quantity, which is jointly therapeutically effective at targeting or preventing proliferative diseases a combination of the invention. In this composition, agent (a) and agent (b) may be administered together, one after the other or separately in one combined unit dosage form or in two separate unit dosage forms. The unit dosage form may also be a fixed combination.

The pharmaceutical compositions for separate administration of agent (a) and agent (b) or for the administration in a fixed combination, i.e., a single galenical composition comprising at least two combination partners (a) and (b), according to the invention may be prepared in a manner known per se and are those suitable for enteral, such as oral or rectal, and parenteral administration to mammals (warm-blooded animals), including humans, comprising a therapeutically effective amount of at least one pharmacologically active combination partner alone, e.g., as indicated above, or in combination with one or more pharmaceutically acceptable carriers or diluents, especially suitable for enteral or parenteral application.

Suitable pharmaceutical compositions contain, e.g., from about 0.1% to about 99.9%, preferably from about 1% to about 60%, of the active ingredient(s). Pharmaceutical preparations for the combination therapy for enteral or parenteral administration are, e.g., those in unit dosage forms, such as sugar-coated tablets, tablets, capsules or suppositories, or ampoules. If not indicated otherwise, these are prepared in a manner known per se, e.g., by means of conventional mixing, granulating, sugar-coating, dissolving or lyophilizing processes. It will be appreciated that the unit content of a combination partner contained in an individual dose of each dosage form need not in itself constitute an effective amount since the necessary effective amount can be reached by administration of a plurality of dosage units.

In particular, a therapeutically effective amount of each of the combination partner of the combination of the invention may be administered simultaneously or sequentially and in any order, and the components may be administered separately or as a fixed combination. For example, the method of preventing or treating proliferative diseases according to the invention may comprise: (i) administration of the first agent (a) in free or pharmaceutically acceptable salt form; and (ii) administration of an agent (b) in free or pharmaceutically acceptable salt form, simultaneously or sequentially in any order, in jointly therapeutically effective amounts, preferably in synergistically effective amounts, e.g., in daily or intermittently dosages corresponding to the amounts described herein. The individual combination partners of the combination of the invention may be administered separately at different times during the course of therapy or concurrently in divided or single combination forms. Furthermore, the term administering also encompasses the use of a pro-drug of a combination partner that convert in vivo to the combination partner as such. The instant invention is therefore to be understood as embracing all such regimens of simultaneous or alternating treatment and the term “administering” is to be interpreted accordingly.

The effective dosage of each of the combination partners employed in the combination of the invention may vary depending on the particular compound or pharmaceutical composition employed, the mode of administration, the condition being treated, the severity of the condition being treated. Thus, the dosage regimen of the combination of the invention is selected in accordance with a variety of factors including the route of administration and the renal and hepatic function of the patient. A clinician or physician of ordinary skill can readily determine and prescribe the effective amount of the single active ingredients required to alleviate, counter or arrest the progress of the condition. Optimal precision in achieving concentration of the active ingredients within the range that yields efficacy without toxicity requires a regimen based on the kinetics of the active ingredients' availability to target sites.

Daily dosages for agent (a) or (b) or will, of course, vary depending on a variety of factors, e.g., the compound chosen, the particular condition to be treated and the desired effect. In general, however, satisfactory results are achieved on administration of agent (a) at daily dosage rates of the order of ca. 0.03 to 5 mg/kg per day, particularly 0.1 to 5 mg/kg per day, e.g. 0.1 to 2.5 mg/kg per day, as a single dose or in divided doses. Agent (a) and agent (b) may be administered by any conventional route, in particular enterally, e.g. orally, e.g. in the form of tablets, capsules, drink solutions or parenterally, e.g. in the form of injectable solutions or suspensions. Suitable unit dosage forms for oral administration comprise from ca. 0.02 to 50 mg active ingredient, usually 0.1 to 30 mg, e.g. agent (a) or (b), together with one or more pharmaceutically acceptable diluents or carriers therefore.

Agent (b) may be administered to a human in a daily dosage range of 0.5 to 1,000 mg. Suitable unit dosage forms for oral administration comprise from ca. 0.1 to 500 mg active ingredient, together with one or more pharmaceutically acceptable diluents or carriers therefore.

The administration of a pharmaceutical combination of the invention results not only in a beneficial effect, e.g., a synergistic therapeutic effect, e.g., with regard to inhibiting the unregulated proliferation of hematological stem cells or slowing down the progression of leukemias, such as CML (chronic myeloid leukemia), ALL (acute lymphocyte leukemia) or AML (acute myeloid leukemia), or the growth of tumors, but also in further surprising beneficial effects, e.g., less side effects, an improved quality of life or a decreased morbidity, compared to a monotherapy applying only one of the pharmaceutically active ingredients used in the combination of the invention.

A further benefit is that lower doses of the active ingredients of the combination of the invention can be used, e.g., that the dosages need not only often be smaller but are also applied less frequently, or can be used in order to diminish the incidence of side effects. This is in accordance with the desires and requirements of the patients to be treated.

Combinations of a pyrimidylaminobenzamide compound and an HSP90 inhibitor may be combined, independently or together, with one or more pharmaceutically acceptable carriers and, optionally, one or more other conventional pharmaceutical adjuvants and administered enterally, e.g., orally, in the form of tablets, capsules, caplets, etc. or parenterally, e.g., intraperitoneally or intravenously, in the form of sterile injectable solutions or suspensions. The enteral and parenteral compositions may be prepared by conventional means.

The combination of a pyrimidylaminobenzamide compound and an HSP90 inhibitor can be used alone or combined with at least one other pharmaceutically active compound for use in these pathologies. These active compounds can be combined in the same pharmaceutical preparation or in the form of combined preparations “kit of parts” in the sense that the combination partners can be dosed independently or by use of different fixed combinations with distinguished amounts of the combination partners, i.e., simultaneously or at different time points. The parts of the kit of parts can then, e.g., be administered simultaneously or chronologically staggered, that is at different time points and with equal or different time intervals for any part of the kit of parts. Non-limiting examples of compounds which can be cited for use in combination with the combination of a pyrimidylaminobenzamide compound and an HSP90 inhibitor are cytotoxic chemotherapy drugs, such as cytosine arabinoside, daunorubicin, doxorubicin, cyclophosphamide, VP-16, or imatinib etc. Further, the combination of a pyrimidylaminobenzamide compound and an HSP90 inhibitor could be combined with other inhibitors of signal transduction or other oncogene-targeted drugs with the expectation that significant synergy would result.

### B. Diseases to be Treated

The term “proliferative disease” includes but is not restricted to tumors, psoriasis, restenosis, sclerodermitis and fibrosis.

The term hematological malignancy, refers in particular to leukemias, especially those expressing BCR-ABL, c-Kit or HSP90, (or those depending on BCR-ABL, c-Kit or HSP90) and includes, but is not limited to, CML and ALL, especially the Philadelphia chromosome positive acute lymphocyte leukemia (Ph+ALL), as well as Imatinib-resistant leukemia. Especially preferred is use of the combinations of the present invention for leukemias, such as CML, ALL or AML. Most especially preferred is use in diseases which show resistance to Imatinib. (Imatinib and is sold under the name Gleevec®)

The term “a solid tumor disease” especially means ovarian cancer, breast cancer, cancer of the colon and generally the gastrointestinal tract, such as gastrointestinal stromal tumors (GISTs), cervical cancer, lung cancer, e.g., small-cell lung cancer and non-small-cell lung cancer, head and neck cancer, bladder cancer, cancer of the prostate or Kaposi's sarcoma.

The combinations according to the invention, that inhibit the protein kinase activities mentioned, especially tyrosine protein kinases mentioned above and below, can therefore be used in the treatment of protein kinase dependent diseases. Protein kinase dependent diseases are especially proliferative diseases, preferably benign or especially malignant tumors (e.g., carcinoma of the kidneys, brain, liver, adrenal glands, bladder, breast, stomach (especially gastric tumors), ovaries, colon, rectum, prostate, pancreas, lungs (especially SCLC), vagina or thyroid, sarcoma, multiple myeloma, glioblastomas and numerous tumors of the neck and head, as well as leukemias); especially colon carcinoma or colorectal adenoma, or a tumor of the neck and head, an epidermal hyperproliferation, especially psoriasis, prostate hyperplasia, a neoplasia, especially of epithelial character, preferably mammary carcinoma, or a leukemia. They are able to bring about the regression of tumors and to prevent the formation of tumor metastases and the growth of (also micro)metastases. In addition they can be used in epidermal hyperproliferation (e.g., psoriasis), in prostate hyperplasia, and in the treatment of neoplasias, especially of epithelial character, e.g., mammary carcinoma. It is also possible to use the combinations of the present invention in the treatment of diseases of the immune system insofar as several or, especially, individual tyrosine protein kinases are involved; furthermore, the combinations of the present invention can be used also in the treatment of diseases of the central or peripheral nervous system where signal transmission by at least one tyrosine protein kinase, especially selected from those mentioned specifically, is involved.

In CML, a reciprocally balanced chromosomal translocation in hematopoietic stem cells (HSCs) produces the BCR-ABL hybrid gene. The latter encodes the oncogenic BCR-ABL fusion protein. Whereas ABL encodes a tightly regulated protein tyrosine kinase, which plays a fundamental role in regulating cell proliferation, adherence and apoptosis, the BCR-ABL fusion gene encodes as constitutively activated kinase, which transforms HSCs to produce a phenotype exhibiting deregulated clonal proliferation, reduced capacity to adhere to the bone marrow stroma and a reduces apoptotic response to mutagenic stimuli, which enable it to accumulate progressively more malignant transformations. The resulting granulocytes fail to develop into mature lymphocytes and are released into the circulation, leading to a deficiency in the mature cells and increased susceptibility to infection. ATP-competitive inhibitors of BCR-ABL have been described which prevent the kinase from activating mitogenic and anti-apoptotic pathways (e.g., P-3 kinase and STAT5), leading to the death of the BCR-ABL phenotype cells and thereby providing an effective therapy against CML. The combinations of the present invention are thus especially appropriate for the therapy of diseases related to its overexpression, especially leukemias, such as leukemias, e.g., CML or ALL.

In a broader sense of the invention, a proliferative disease includes hyperproliferative conditions, such as leukemias, hyperplasias, fibrosis (especially pulmonary, but also other types of fibrosis, such as renal fibrosis), angiogenesis, psoriasis, atherosclerosis and smooth muscle proliferation in the blood vessels, such as stenosis or restenosis following angioplasty. In another aspect the combinations of the present invention could be used to treat arthritis.

Combinations of the present invention can also be used to treat or prevent fibrogenic disorders, such as scleroderma (systemic sclerosis); diseases associated with protein aggregation and amyloid formation such as Huntington's disease; inhibition of the replication of hepatitis C virus and treating hepatitis C virus; treating tumors associated with viral infection, such as human papilloma virus; and inhibiting viruses dependent of heat-shock proteins.

The combinations of the present invention primarily inhibit the growth of blood vessels and are thus, e.g., effective against a number of diseases associated with deregulated angiogenesis, especially diseases caused by ocular neovascularisation, especially retinopathies, such as diabetic retinopathy or age-related macula degeneration, psoriasis, hemangioblastoma, such as hemangioma, mesangial cell proliferative disorders, such as chronic or acute renal diseases, e.g., diabetic nephropathy, malignant nephrosclerosis, thrombotic microangiopathy syndromes or transplant rejection, or especially inflammatory renal disease, such as glomerulonephritis, especially mesangioproliferative glomerulonephritis, hemolytic-uremic syndrome, diabetic nephropathy, hypertensive nephrosclerosis, atheroma, arterial restenosis, autoimmune diseases, diabetes, endometriosis, chronic asthma, and especially neoplastic diseases (solid tumors, but also leukemias and other hematological malignancies), such as especially breast cancer, cancer of the colon, lung cancer (especially small-cell lung cancer), cancer of the prostate or Kaposi's sarcoma. Combinations of the present invention inhibit the growth of tumors and are especially suited to preventing the metastatic spread of tumors and the growth of micrometastases.

We claim:

1. A pharmaceutical combination comprising:
a) a pyrimidylaminobenzamide compound of formula (I); and
b) at least one HSP90 inhibitor.

2. A pharmaceutical combination according to claim 1 wherein agent a) is selected from 4-methyl-3-[[4-(3-pyridinyl)-2-pyrimidinyl]amino]-N-[5-(4-methyl-1H-imidazol-1-yl)-3-(trifluoromethyl)phenyl]benzamide of formula (II):

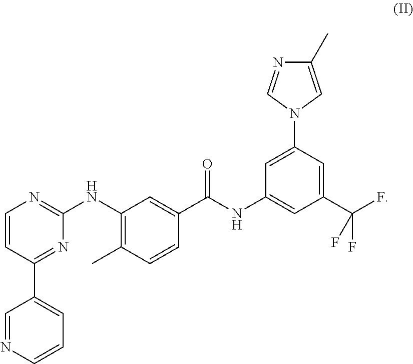 [SMILES: Cc1cn(-c2cc(NC(=O)c3ccc(C)c(Nc4nccc(-c5cccnc5)n4)c3)cc(C(F)(F)F)c2)cn1]

3. A pharmaceutical combination according to claim 2 wherein agent b) is selected from geldanamycin derivatives, 17-allylamino-17-demethoxygeldanamycin (17-AAG), geldanamycin-related compounds, Radicicol, a compound of formula (III) and a compound of formula (IV).

4. Use of a pharmaceutical combination according to claim 1 for the preparation of a medicament for the treatment or prevention a proliferative disease.

5. A use according to claim 4 wherein the disease is chronic myeloid leukemia, acute lymphocyte leukemia, acute myeloid leukemia or gastrointestinal stromal tumors.

6. A method for treating or preventing a proliferative disease in a subject in need thereof, comprising co-administration to said subject, e.g. concomitantly or in sequence, of a therapeutically effective amount of at least one HSP90 inhibitor and a pyrimidylaminobenzamide compound of formula (I).

7. A method for treating chronic myeloid leukemia, acute lymphocyte leukemia, acute myeloid leukemia or gastrointestinal stromal tumors comprising administering a combination of an HSP90 inhibitor and a pyrimidylaminobenzamide compound of formula (I).

8. A method for treating chronic myeloid leukemia, acute lymphocyte leukemia, acute myeloid leukemia or gastrointestinal stromal tumors comprising administering a combination of an HSP90 inhibitor and 4-methyl-3-[[4-(3-pyridinyl)-2-pyrimidinyl]amino]-N-[5-(4-methyl-1H-imidazol-1-yl)-3-(trifluoromethyl)phenyl]benzamide.

9. A method for treating chronic myeloid leukemia, acute lymphocyte leukemia, acute myeloid leukemia or gastrointestinal stromal tumors comprising administering a combination of an HSP90 inhibitor and a pyrimidylaminobenzamide compound of formula (I), wherein the HSP90 inhibitor is 17-allylamino-17-demethoxygeldanamycin.
